(19) United States
(12) Patent Application Publication
(10) Pub. No.: US 2004/0215107 A1
(43) Pub. Date: Oct. 28, 2004

(54) Blood collection device with a holder having a cannula for venipuncture

(75) Inventors: Walter Sarstedt, Numbrecht (DE); Horst Farber, Numbrecht (DE)

Correspondence Address: THE FIRM OF KARL F ROSS, 5676 RIVERDALE AVENUE, PO BOX 900, RIVERDALE (BRONX), NY, 10471-0900, US

(73) Assignee: SARSTEDT AG & Co.

(21) Appl. No.: 10/768,386

(22) Filed: Jan. 29, 2004

(30) Foreign Application Priority Data
Jan. 31, 2003 (DE) 20301575.4

(51) Int. Cl. A61B 5/00

(52) U.S. Cl. 600/576

(57) ABSTRACT

A blood collection device has a holder having a forwardly extending sharp-edged cannula capable of venipuncture and a rearwardly extending cannula. A guide sleeve into which a blood-collection tube is insertable from one end, at an opposite end, threadedly receives the holder whereby the tube can be filled with blood conducted by the rearwardly extending cannula into the device. A closed end elastic tubular valve rubber sheath surrounding and hugging the rearwardly extending cannula is shape locked and force fitted in said guide sleeve.

### FIELD OF THE INVENTION

The present invention relates to a blood collection device of the type in which a guide sleeve, also commonly referred to as a holder, is open at one end to receive a blood-collection tube and at the other end receives the cannula and the cannula holder.

### BACKGROUND OF THE INVENTION

U.S. Pat. No. 3,585,984 describes a blood collection device in which the holder is formed with a cannula in the form of a hollow needle with a sharpened edge designed to penetrate into a vein and draw blood therefrom. The cannula opens at the other end of the holder into a hose-like closed elastic tubular valve-rubber member. Air is present between the cannula and this valve-rubber sheath. The cannula can have a rearward extension extending into this sheath which can have a thickened head enabling it to be fitted over the cannula and frictionally held between the cannula and the guide sleeve.

In DE 19 617 000, the valve-rubber sheath is clamped against the cannula by a formation of the cannula holder. In DE 1 812 742, the guide sleeve is fixed to the cannula and the valve-rubber member is frictionally held in the guide sleeve.

The cannula may be a double ended cannula which can have its inner end, i.e. the end received within the guide tube and extending opposite the end of the cannula which is to penetrate the vein, surrounded by the valve-rubber sheath. The reference to a “valve-rubber sheath” is here intended to indicate that sheath can be penetrated by the inner needle end as the latter punctures the cap of the collection tube which is inserted into the guide sleeve.

In DE-U 8 016 927, a cylindrical guide sleeve has an external conical formation onto which the holder provided with the sharpened hollow needle or cannula is mounted. This system has the advantage that it allows a Luer cannula to be used as an option.

In D 29 03 167, a blood collection device of the safety type for multiple collections is described in which the connection with the blood receiving tube imparts an accordion pleating to the valve-rubber sheath and again air can be present in the radial gap between the sheath and the inner of the double cannula. The valve-rubber sheath here has over its entire length an outer diameter which is greater than the outer diameter of the inner portion of the cannula.

The earlier blood collection devices have a variety of drawbacks, in part arising because there may be unavoidable friction between the needle and the valve-rubber sheath (see DE 8 016 927) or some other delay in the expansion of the accordion pleated sheath before it returns to its original position to seal the inner needle prior to the next sample taking.

Thus the valve-rubber sheath may not fully and completely seal the inner needle with sufficient speed after one sample has been drawn and before a second tube has been thrust onto that needle.

Mention may be made of the fact that DE 1 812 742 does show a shoulder for the valve-rubber sheath which lies against an inner shoulder of the holder, but nevertheless here that sheath is held in place at least in part by friction.

The valve-rubber sheath of DE 2 903 167 has at its open end an outwardly extending shoulder which is fitted into an annular groove of the end holder. Nevertheless, here as well there is a loose seat in the holder which may create problems with respect to mounting of the valve-rubber member and the fitting of the valve-rubber member in the annular groove. Mention may also be made of a number of other systems for fastening the valve-rubber sheath in place. For example, EP 0 678 279 provides the valve-rubber sheath with a collar which is suspended on an annular groove edge within the holder and is s secured by a plastic member in place.

EP 0 619 916 and WO-A 95/16395 show other techniques. Systems are described which use an adhesive or other bonding process and may involve a baking step. In some of these arrangements, sterilization of the unit can damage the attachment.

### OBJECT OF THE INVENTION

It is, therefore, the principal object of the present invention to provide an improved blood collection device and particularly, a blood collection device with improved attachment of the valve-rubber sheath.

Another object of the invention is to provide an improved blood collection arrangement whereby drawbacks of earlier systems are avoided.

### SUMMARY OF THE INVENTION

These objects and others which will become apparent hereinafter are attained, in accordance with the invention by mounting the valve-rubber sheath so that it is shape-locked and force-locked in the guide housing. The term shape-locked and force-fitted in the guide sleeve means, according to a feature of the invention, that the open end of the elastic tubular valve rubber sheath has an outwardly extending shoulder which can have a surface which is turned toward the closed end and lies in a plane perpendicular to the axis of that sheath or is inclined to that axis, i.e. of frustoconical shape and converges toward the closed end of that sheath. According to the invention, that surface lies against a similarly shaped and inwardly extending surface formed unitarily on the end wall of the guide sleeve and preferably on a frustoconical boss projecting toward the interior of that guide sleeve and is in alignment with the threaded portion of the outwardly extending boss on that end wall of the guide sleeve in which the cannula holder is threaded, the cannula holder having a shoulder bearing upon the open end of the valve-rubber sheath. In other words, the head of the sheath is clamped by the end wall or shoulder of the threaded portion of the holder against the aforementioned surface on that end wall of the guide sleeve itself. Since the fastening of the valve rubber sheath is directly between the shoulder of the cannula holder and the guide sleeve wall without any adhesive or other bonding material, even with the use of aggressive sterilization agents, for example, ozone, there is no loosening of the attachment. Furthermore, there is no requirement for any substantial compressive loading of the valve-rubber sheath and, instead, in the event of sterilization, the memory effect of the valve-rubber sheath can be brought into play to restore an original seal around the inwardly extending cannula. Furthermore, that sheath can hug the inwardly extending cannula over the entire length thereof so that there no longer is an air space between the sheath and the inwardly extending needle.

Thus at its open end, the valve-rubber sheath is suspended by its shoulder in the guide sleeve directly and at this end or shoulder, the head of this valve-rubber sheath is clamped in place by the cannula holder. When matching conical surfaces are provided between the open end of the valve-rubber sheath and the guide sleeve, the two cones need not engage with any substantial prestress and merely the screwing of the holder into the guide sleeve can suffice to hold the valve-rubber sheath in place.

### BRIEF DESCRIPTION OF THE DRAWING

The above and other objects, features, and advantages will become more readily apparent from the following description, reference being made to the accompanying drawing in which:

FIG. 1 is an axial cross sectional view of one embodiment of a device in accordance with the invention; and

FIG. 2 is a view similar to FIG. 1 illustrating another embodiment of the invention.

### SPECIFIC DESCRIPTION

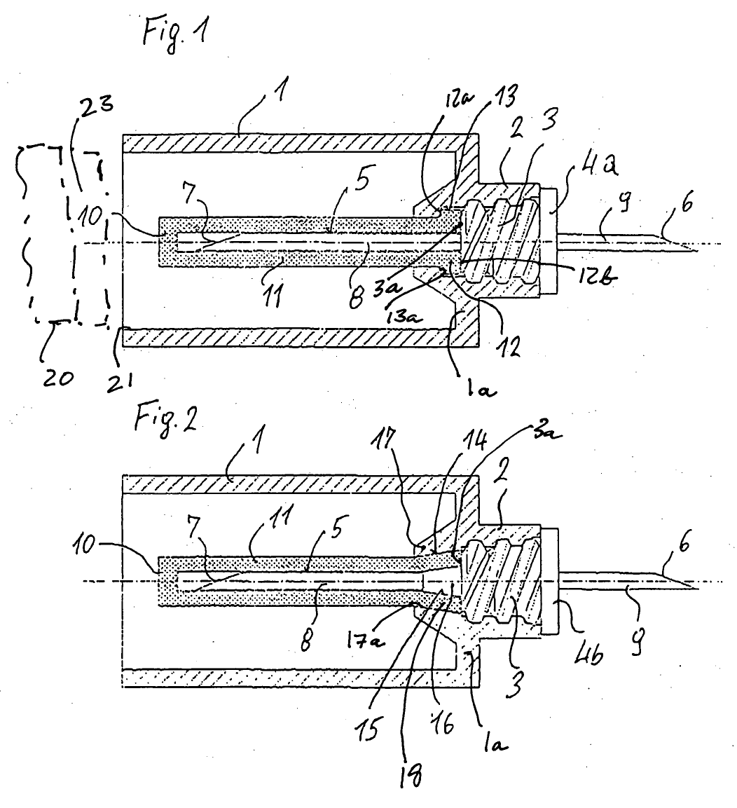

In FIGS. 1 and 2, there are shown two embodiments of a blood collection device which may be generally of the type marked under the trademark Vacutainer® and wherein a guide sleeve 1 is formed with a closed end formed by an end wall la provided with an internally threaded boss or collar 2 which receives the threaded portion 3 of a cannula holder 4a or 4b. The cannula holder 4a has a venipuncture needle 9 extending outwardly and having a sharpened edge 6 adapted to pierce the vein. The needle or cannula 9 is hollow and communicates with an inner cannula or needle 8, extending from the holder 4a into the guide sleeve 1 and provide with a sharpened end 7 therein. The result is a so-called double cannula as designated at 5.

When the needle 9 is inserted into the vein, a collection tube 20 is inserted into the open end 21 of the guide sleeve until its self sealing cap 23 is pierced by the end 7 of the cannula 6.

Surrounding the inwardly extending cannula 8 is a valve-rubber sheath 11 with a closed end 10, likewise pierced by the needle 7, 8, but which selfseals, once the tube 20 is withdrawn after filling.

At the opposite end of the valve-rubber sheath 10, i.e. its open end, a shoulder 13 is provided which is received in a chamber 13 at the end wall la of the guide sleeve 1 so that the surface 12a of the shoulder, which lies perpendicular to the axis of the sheath, directly abuts a wall 13a of the compartment 13 which likewise is perpendicular to the axis. The end 12b of the shoulder 12 is engaged by an end 3a of the threaded portion 3 so that the head or shoulder 12 is held between the surfaces 3a and 13a in a shape-locked and force-fitted engagement.

The same principle applies in FIG. 2 wherein, however, the conically inwardly extending projection 17 on the end wall la has an internal conical surface 17a against which the outer cone 14 of the head 18 of the valve-rubber sheath 11 can engage. An inner conical surface 15 of the head 18 matches the conical formation 16 at the junction of the inwardly extending needle 8 and the threaded portion 3. The conical surfaces are pressed together by the end 3a of the threaded portion 3. Here, as well, there is an axial clamping of the valve-rubber sheath between surfaces 17a and 3a in shape locking and force fitting the s valve-rubber sheath in place.

What is claimed is:

1. A blood collection device comprising:
a holder having a forwardly extending sharp-edged cannula capable of venipuncture extending from said holder and a rearwardly extending cannula;
a guide sleeve into which a blood-collection tube is insertable from one end and, at an opposite end, threadedly receiving said holder whereby said tube can be filled with blood conducted by said rearwardly extending cannula into the device; and
a closed end elastic tubular valve rubber sheath surrounding and hugging said rearwardly extending cannula and shape locked and force fitted in said guide sleeve.

2. The blood collection device defined in claim 1 wherein said guide sleeve is formed unitarily with end wall at said opposite end, a frustoconical inwardly extending boss on said end wall, and an outwardly internally threaded collar on said end wall aligned with said boss, said valve rubber sheath having a head at an open end of said sheath formed with a surface juxtaposed with a surface of said boss whereby said sheath is clamped by a threaded portion of said holder against said surface of said boss.

3. The blood collection device defined in claim 2 wherein said surfaces lie in planes perpendicular to an axis of said valve rubber sheath.

4. The blood collection device defined in claim 2 wherein said surfaces are frustoconical surfaces tapering toward a closed end of said sheath.

5. The blood collection device defined in claim 4 wherein said holder is formed with an externally frustoconical portion adjacent said threaded portion and engaging an internal frustoconical portion of said head of said sheath.
